(19) United States
(12) Patent Application Publication
(10) Pub. No.: US 2025/0197442 A1
(43) Pub. Date: Jun. 19, 2025

(54) Peptide Synthesis in Aqueous Solution

(71) Applicant: Yong Ma, San Pablo, CA (US)

(72) Inventor: Yong Ma, San Pablo, CA (US)

(21) Appl. No.: 18/583,562

(22) Filed: Feb. 21, 2024

Related U.S. Application Data
(60) Provisional application No. 63/610,627, filed on Dec. 15, 2023.

(51) Int. Cl. C07K 1/04; C07C 229/08; C07C 309/01; C07K 1/06

(52) U.S. Cl. CPC C07K 1/042; C07C 229/08; C07C 309/01; C07K 1/063

(57) ABSTRACT

Four linker molecules were designed to attach peptides to various aqueous-compatible polymer solid phases for Solid Phase Peptide Synthesis (SPPS) in aqueous solutions. Linker molecule A is utilized to attach peptides to an anionic exchange resin in an aqueous solution, and linker molecule B is employed for binding peptides to a cationic exchange resin in an aqueous solution. Both anionic and cationic exchange resins are reusable once the peptide synthesis process is completed.

Linker molecule C is utilized to connect the peptide to an amino resin, whereas linker molecule D is employed to bind the peptide to a carboxyl resin. Additionally, linker molecule C can serve as a precursor to linker molecule A, and linker molecule D can function as a precursor to linker molecule B.

In all four linker molecules, the amino acid (AA) is linked to solid phases through a benzylic ester bond, which can be cleaved under acidic conditions to release the peptide chain at the end of synthesis.

A procedure for dissolving hydrophobic molecules was developed to improve the solubility of hydrophobic compounds, specifically focusing on dissolving Fmoc-protected Amino Acids (Fmoc-AA) in an aqueous solution to facilitate Solid Phase Peptide Synthesis (SPPS) in aqueous conditions.

### FIELD OF THE INVENTION

The present invention relates generally to peptide synthesis. Particularly, the present invention comprises a method for conducting solid-phase peptide synthesis in an aqueous solution and compositions of linker molecules used for conducting solid-phase peptide synthesis in an aqueous solution.

### BACKGROUND OF THE INVENTION

Solid-phase peptide synthesis (SPPS) is a method for peptide synthesis. In SPPS, a peptide is tethered by its C-terminus to an insoluble polymer, and the peptide is subsequently constructed through the sequential addition of protected amino acids. Solid-phase peptide synthesis (SPPS) is predominantly carried out in organic solvents within the current peptide industry.

Dimethylformamide (DMF) and dichloromethane (CH2Cl2), known for their serious toxicity concerns and waste solvent generation, are widely utilized in organic solvents for peptide synthesis. There is an urgent demand for the advancement of more environmentally friendly and safer protocols in the realm of peptide synthesis.

Aqueous-phase peptide synthesis typically employs protected amino acids, coupling reagents, and other essential additives that are soluble or dispersible in water. Several strategies have been devised to facilitate peptide bond formation and safeguard the reactive functional groups of amino acids in an aqueous environment.

Recently, certain researchers have suggested that propylene carbonate, recognized for its environmentally friendly and polar properties, could effectively replace dichloromethane and DMF in both solution and solid-phase peptide synthesis.

Another novel technology has surfaced in peptide synthesis, featuring a tandem deprotection/coupling sequence designed for solution-phase peptide synthesis in water. This process operates under micellar catalysis conditions, employing the unique surfactant TPGS-750-M.

Another invention introduces a method for peptide synthesis, involving the steps of condensing an N-Fmoc protected amino acid with a peptide possessing C-terminal protection. This process employs a carrier that crystallizes in response to alterations in the solvent composition.

One common approach in aqueous-phase peptide synthesis is the use of water-soluble coupling reagents, such as water-soluble carbodiimides or peptide coupling agents that can function efficiently in an aqueous medium. Additionally, protecting groups that are compatible with water-based conditions are employed to selectively mask the functional groups that could react undesirably during peptide bond formation.

Overall, aqueous-phase peptide synthesis is an active area of research and development, with ongoing efforts to improve the efficiency, scalability, and sustainability of peptide synthesis methods. The use of water as a primary solvent provides several advantages in terms of safety, environmental impact, and compatibility with downstream applications, making it an attractive option for peptide synthesis.

This invention's objective is to transition from the use of organic solvents to an aqueous system while retaining the current SPPS practices in organic solvents as much as possible. This conversion entails minimal changes, with no significant differences from the organic system, except for the peptide linker molecules and hydrophilic solid resins which are commercially available. This approach aims to make the transition more readily acceptable for the industry.

Another objective of this invention is to offer a peptide synthesis method mostly within an aqueous solvent, encompassing Fmoc deprotection, coupling, and cleavage processes, thereby eliminating the need for organic solvents in these stages.

### SUMMARY OF THE INVENTION

The present invention generally pertains to, and more specifically focus on, the utilization of specifically designed linker molecules to attach peptides to hydrophilic resins in Solid-Phase Peptide Synthesis (SPPS) within an aqueous environment, while Fmoc-protected Amino Acids (Fmoc AAs) serving as building blocks to facilitate the peptide sequence assembly within the aqueous solution. Specifically designed four linkage molecules are developed to establish bonds between the peptides and the solid phases in the aqueous phase. A tailored procedure has been devised to enhance the solubility of Fmoc AAs in the aqueous phase. This innovative approach aims to minimize the utilization of organic solvents in peptide synthesis, thereby contributing to greener and more sustainable synthetic methodologies.

Four novel linker molecules were developed for attaching peptides to diverse aqueous-compatible polymer solid phases in aqueous solutions. Linker molecule A is used for attaching peptides to anionic exchange resins, while linker molecule B is employed to bind peptides to cationic exchange resins. Both anionic and cationic exchange resins can be reused after the peptide synthesis process is completed. Linker molecule C is employed to link the peptides to amino resins, while linker molecule D is used to bind the peptide to a carboxyl resins. The four linker molecules have structures are illustrated below.

Linker molecule A has a general structure of AA-CH2-Ph-Rx-SO₃⁻ (as illustrated in FIG. 1). The sulfonate —SO₃⁻ provides ionic bond to an anionic exchange resin in aqueous solution, Linker molecule A is used for attaching peptides to an anionic exchange resin. The anionic resin can be regenerated once the synthesis is completed. AA (initial Amino Acid) group linking to Ph group through structure —COO—CH2-Ph-which is capable of being split under strong acidic conditions, Ph is benzene ring. The Rx group offers diverse options, including but not limited to the methoxyphenyl group (—O—CH2-Ph-), methylene group (—CH2-), carboxylate group (—OOC—), carboxymethylene group (—OOC—CH2-), amino group (—NH—), and ether linkage group (—O—). These chemical groups are utilized individually or in combinations of two or more groups in Rx structure. The Rx group comprises two or more carbon atoms, preferably 5 to 25 carbon atoms. The Rx group is a “spacer” to provide space between Ph and COOH, the spacer makes linker molecule flexible, Rx also facilitates binding Fmoc-AA-CH2-Ph- and —COOH group. This Rx group designed imparts flexible structure within the linker molecule, providing easier way to build connection between benzene group and sulfonate group.

Linker molecule B is employed to bind peptides to a cationic exchange resin, Linker molecule B has a general structure of AA-CH2-Ph-Rx-N(CH₃)₃⁺ (as illustrated in FIG. 2). The trimethylammonium group-N(CH₃)₃⁺ provides ionic bond to cationic exchange resin in aqueous solution. The Amino Acids (AA) group linking through structure —COO—CH2-Ph-which is capable of being split under strong acidic conditions. The cationic exchange resin can be regenerated once the synthesis is completed. The Rx group offers diverse options, including but not limited to the methoxyphenyl group (—O—CH2-Ph-), methylene group (—CH2-), carboxylate group (—OOC—), carboxymethylene group (—OOC—CH₂—), amino group (—NH—), and ether linkage group (—O—). These chemical groups are utilized individually or in combinations of two or more groups in Rx structure.

The linker molecule C possesses a general structure of Fmoc-AA-CH₂-Ph-Rx-COOH (as illustrated in FIG. 3), Fmoc (Fluorenylmethoxycarbonyl) is protecting group for AA (Amino Acid). The Amino Acids (AA) group linking through structure —COO—CH₂-Ph-which is capable of being split under strong acidic conditions. The carboxyl group can form a covalent bond with the amino resin (as shown in FIG. 5) as follows:
Fmoc-AA-CH₂-Ph-Rx-COOH+amino-resin+coupling agent→Fmoc-AA-CH₂-Ph-Rx-COO—NH-resin

The Rx group offers diverse options, including but not limited to the methoxyphenyl group (—O—CH2-Ph-), methylene group (—CH2-), carboxylate group (—OOC—), carboxymethylene group (—OOC—CH2-), amino group (—NH—), and ether linkage group (—O—). These chemical groups are utilized individually or in combinations of two or more groups in Rx structure. The Rx group comprises two or more carbon atoms, preferably 5 to 25 carbon atoms. The Rx group is a “spacer” to provide space between Ph and COOH, the spacer makes linker molecule flexible, Rx also facilitates binding Fmoc-AA-CH2-Ph- and —COOH group.

In addition, linker molecule C can be used as a precursor of the linker molecule A. Coupling linker molecule C with Taurine using a coupling agent such as EDC results in the formation of liner molecule A (as shown in FIG. 7). Subsequently, the Fmoc can be removed in a basic solution.
Fmoc-AA-CH2-Ph-Rx-COOH+NH2-R—SO₃⁻→Fmoc-AA-CH2-Ph-Rx-COO—NH-Rx-SO₃⁻, (FIG. 7)

Linker molecule D possesses a general structure of Fmoc-AA-CH2-Ph-Rx-NH2 (as illustrated in FIG. 4), The —NH2 group is used to form a covalent bond with the carboxyl resin (example: carboxyl methyl cellulose) (as illustrated in FIG. 6)

Linker molecule D+carboxyl resin+coupling agent→Fmoc-AA-CH2-Ph-Rx-NH-carboxyl-resin

In addition, molecule D can be used as a precursor for linker molecule B (as illustrated in FIG. 8):
Fmoc-AA-CH2-Ph-R—NH2+3-carboxypropyl) trimethylammonium→linker molecule B

Coupling linker molecule D with 3-carboxypropyl) trimethylammonium using a coupling agent such as EDC results in the formation of linker molecule B (as shown in FIG. 8). Subsequently, the Fmoc can be removed in a basic solution.

This invention provides a scheme for synthesizing peptides via solid-phase peptide synthesis (SPPS) in an aqueous solution. Similar to SPPS in organic solvents, Fmoc-protected amino acids also serve as the fundamental building blocks in this invention. Fmoc-protected amino acids (Fmoc AAs) are extensively utilized in solid-phase peptide synthesis (SPPS) in organic solvent. Nevertheless, Fmoc AAs are inherently insoluble in water. This invention presents a method for dissolving Fmoc AAs in water, enabling the synthesis of peptides in an aqueous phase while still using Fmoc AAs as the building blocks. By doing so, it allows the transition of many practices from traditional organic solvent-based Solid-Phase Peptide Synthesis (SPPS) to peptide synthesis in an aqueous environment.

A Process for Enhancing the Solubility of Hydrophobic Molecules in Aqueous Solution. This invention devised a scheme to render certain hydrophobic molecules soluble into aqueous solution, especially useful for dissolving Fmoc-AA in aqueous solution (as illustrated FIG. 9). It is summarized as follows. First, a non-ionic surfactant is dissolved in a small amount of a water-miscible organic solvent to create solution A, and then secondly, Fmoc amino acid (Fmoc-AA) is dissolved in a small portion of a water-miscible organic, creating solution B., Solution A is thoroughly mixed with Solution B to form even mixture. The resulting mixture is then vigorously added to an aqueous solution. The solubility of Fmoc-AA can be reached to 1-15% in aqueous solution. Solubility of Fmoc-AA may be various depending on solubility of AA in water solution and temperature, for example AA such as Glycine, Fmoc-Glycine needs much less surfactant than Fmoc-Phe to get soluble into water (as illustrated in FIG. 11).

After attachment of the initial amino acids to solid resins via linker molecules, peptide synthesis is achieved by introducing additional Fmoc-AA that is the next amino acid to be added to the peptide chain, the additional Fmoc-AA is dissolved in an aqueous solution by using the process for enhancing the solubility of hydrophobic molecules in aqueous. Subsequently, a coupling agent like EDC is used to couple the Fmoc-AA to the previous AA which is bond to the solid resin.

De-protection, coupling, and cleavage can be achieved in an aqueous solution. The Fmoc de-protection can be accomplished using a base solution in 10% Tween 80 at pH of 9.5-10.5. For example, 0.1% NaOH or 0.3% mono-amine solution can be used to remove Fmoc from peptide chain. Coupling can be conducted by EDC at pH=4-5 in 10% T ween 80 solution. After peptide synthesis finished, acidic solution such as 0.1% HCl or 0.1% H3PO4 can be employed to cleave the peptide chain off from the solid phase.

### BRIEF DESCRIPTION OF THE DRAWINGS

FIG. 1 is a diagram describing the chemical structure of linker molecule A.

FIG. 2 is a diagram describing the chemical structure of linker molecule B.

FIG. 3 is a diagram describing the chemical structure of linker molecule C.

FIG. 4 is a diagram describing the chemical structure of linker molecule D.

FIG. 5 is a diagram describing linker molecule C binding to solid phase amino resins.

FIG. 6 is a diagram describing linker molecule D binding to solid phase carboxyl resins.

FIG. 7 is a diagram describing linker molecule A made from linker molecule C.

FIG. 8 is a diagram describing linker molecule B made from linker molecule D.

FIG. 9 is a diagram of dissolving hydrophobic molecules in aqueous solution.

FIG. 10 is a flow chart of solid-phase peptide synthesis in aqueous solution.

FIG. 11 is a diagram describing Fmoc-Gly and Fmoc-Phe solubility testing results.

### DETAILED DESCRIPTION OF THE INVENTION

All illustrations of the drawings are for the purpose of describing selected versions of the present invention and are not intended to limit the scope of the present invention.

Solid-phase peptide synthesis (SPPS) in organic solvents is currently the most widely used method in the industry, often requiring significant amounts of organic solvents. Our primary objective is to transition from traditional SPPS in organic solvents to an aqueous solution, while preserving the established methodology of traditional SPPS as much as possible in order to make SPPS in aqueous solution easy acceptable by the peptide industry.

We have developed four linker molecules designed for connecting peptides on various water-compatible solid phases. These solid phases include, but are not limited to, cationic resins, anionic resins, carboxymethyl cellulose resins, and amino PEG resins-all readily available commercially. The goal is to streamline solid-phase peptide synthesis (SPPS) in aqueous environments.

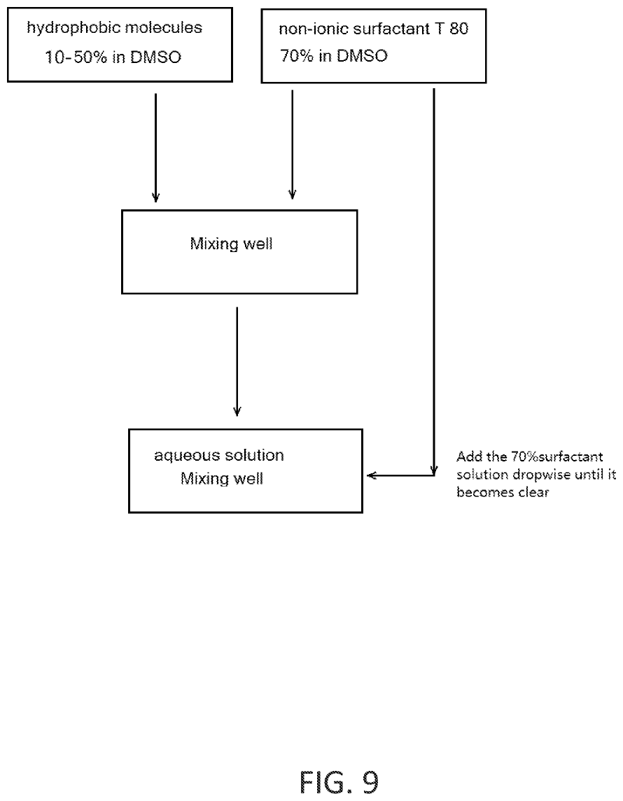

Fmoc protected Amino Acids (Fmoc-AA) are most common building block in SPPS in organic solvents, in order to use Fmoc-AA in SPPS in aqueous solution, it is necessary to make Fmoc-AA soluble in aqueous solution. A process for dissolving Fmoc-AA in Aqueous Solution was developed and it is summarized as follows (FIG. 9). Firstly, a non-ionic surfactant is dissolved in a small portion of a water-miscible organic solvent, creating Solution A. Secondly, FAA is dissolved in another small portion of a water-miscible organic solvent, creating Solution B. These two solutions, Solution A and Solution B, are thoroughly mixed to create a homogeneous mixture. This mixture is then added vigorously to an aqueous solvent. As a result, the Fmoc Amino Acid successfully dissolves in the aqueous phase for solid-phase peptide synthesis (SPPS) in an aqueous solution.

In the present invention, four linker molecules were developed for attaching peptides to diverse aqueous-compatible polymer solid phases in aqueous solutions. Linker molecule A is used for attaching peptides to an anionic exchange resin, while linker molecule B is employed to bind peptides to a cationic exchange resin. Both anionic and cationic exchange resins can be reused after the peptide synthesis process is completed. Linker molecule C is employed to link the peptide to an amino resin, while linker molecule D is used to bind the peptide to a carboxyl resin.

In traditional solid-phase peptide synthesis (SPPS) in organic solvents, Wang Resin is widely favored for its effectiveness. The linker molecule within Wang Resin offers a reversible connection between the peptide chain and the solid support (resin). In Wang Resin, the linker molecule is tethered to the resin via a phenyl ether bond, while the amino acid (AA) is typically linked to the linker molecule through a benzylic ester bond. Trifluoroacetic acid is employed to cleave the ester bond in the linker molecule, thereby releasing the synthesized peptide at the end of the peptide synthesis process.

### Linker Molecule A

In this invention, the structure of linker molecule A, the amino acid (AA) is also linked to the linker molecule through a benzylic ester bond, that resembles of linker molecule in Wang Resin. The primary distinction lies in the fact that linker molecule in Wang Resin is affixed to the solid phase through a chemical bond. Linker molecule A is attached to a solid resin through an ionic bond in an aqueous solution. Linker molecule A is cleavable under acidic conditions to release the synthesized peptide upon the completion of peptide synthesis.

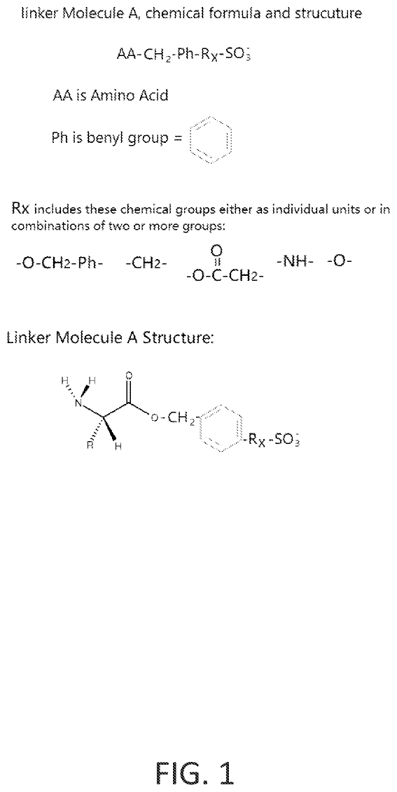

Linker molecule A is specially designed for attaching peptides to anionic exchange resins, linker Molecule A provide linkage between peptide and solid phase through ionic bond, it has a general structure of AA-CH2-Ph-Rx-anionic group. The anionic group includes, but is not limited to, sulfonate and phosphonate groups, preferably sulfonate, its structure likes this: AA-CH2-Ph-Rx-SO₃⁻. Ph represents the benzene group, AA denotes the initial amino acid in the peptide, and —SO₃⁻ signifies the sulfonate group. This sulfonate group is for forming ionic bonds, which enable the linker molecule A to bind the anion exchange resin in aqueous solutions. linker molecule A has general structure as follows:
AA-CH2-Ph-Rx-SO₃⁻(FIG. 1)

Here, the sulfonate —SO₃ provides ionic binding ability in aqueous solution to bind an anionic exchange resin. AA is the first Amino Acid of peptide to be synthesized. The carboxyl group of the amino acid (AA) is linked to the phenyl group through a benzylic ester (—COO—CH2-Ph-). This linkage can be selectively cleaved under acidic conditions at the end of the peptide synthesis process. Consequently, the peptide will be liberated from linker molecule A in this acidic environment. Anion exchange resins can be effectively reused through standard anion resin regeneration procedures.

The Rx group offers diverse options, including but not limited to the methoxyphenyl group (—O—CH2-Ph-), methylene group (—CH2—), carboxylate group (—OOC—), carboxymethylene group (—OOC—CH2—), amino group (—NH—), and ether linkage group (—O—). These chemical groups are utilized individually or in combinations of two or more groups in Rx structure. This Rx designed imparts flexible structure within the linker molecule, providing easier way to build connection between benzene group and sulfonate group. The Rx group comprises two or more carbon atoms, preferably 5 to 25 carbon atoms. The Rx group is a “spacer” to provide space between Ph and COOH, the spacer makes linker molecule flexible, Rx also facilitates binding Fmoc-AA-CH2-Ph- and —COOH group.

### Linker Molecule B

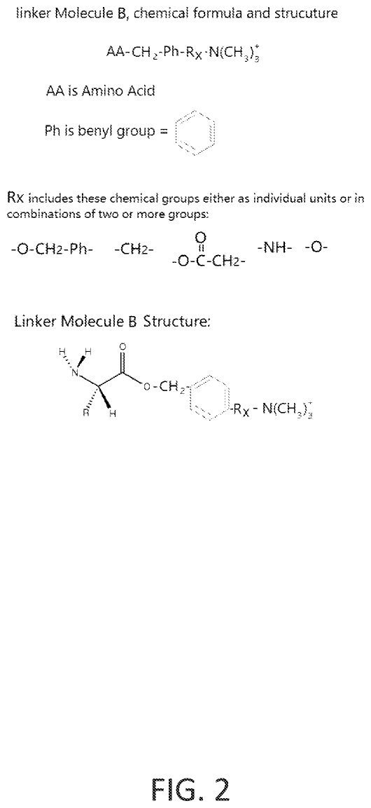

Linker Molecule B is specifically engineered to facilitate the connection between a peptide and a cationic exchange resin in an aqueous solvent. Linker Molecule B bears a resemblance to Linker Molecule A, with the key distinction being the presence of a cationic functional group. It has a general structure of AA-CH2-Ph-Rx-cationic group, in this context, the cationic group may include, but not limited to —N(CH3)₃⁺, Linker molecule B has a structure as follows:
AA-CH2-Ph-Rx-N(CH3)₃⁺(FIG. 2)

AA denotes the initial amino acid in the peptide, and Ph denotes a benzene group. Similar to Linker Molecule A, AA is connected to the phenyl group through a benzylic ester (—COO—CH2-Ph-). Linker Molecule B can bind to cationic exchange resins in an aqueous solution. At the end of the peptide synthesis, the peptide can be separated from Linker Molecule B under acidic conditions. Cationic exchange resins can be efficiently regenerated using standard procedures for regeneration of cationic exchange resins.

The Rx group includes but not limited to the methoxyphenyl group (—O—CH2-Ph-), methylene group (—CH2-), carboxylate group (—OOC—), carboxymethylene group (—OOC—CH2-), amino group (—NH—), and ether linkage group (—O—). These chemical groups are utilized individually or in combinations of two or more groups in Rx structure. The Rx group comprises two or more carbon atoms, preferably 5 to 25 carbon atoms. The Rx group is a “spacer” to provide space between Ph and COOH, the spacer makes linker molecule flexible, Rx also facilitates binding Fmoc-AA-CH2-Ph- and —COOH group. This Rx designed imparts flexible structure within the linker molecule, providing easier way to build connection between benzene group and trimethylammonium group (—N (CH3)₃⁺).

### Linker Molecule C

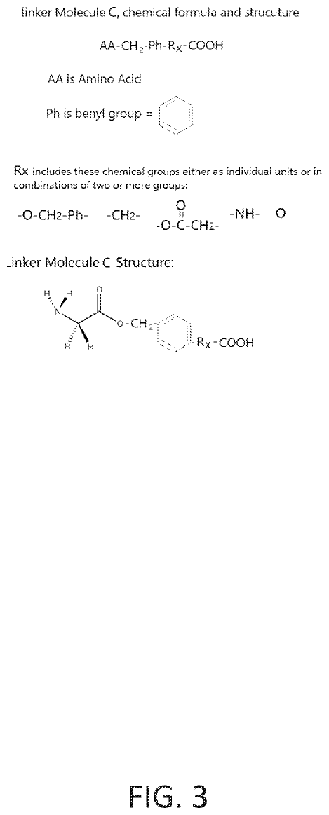

The linker molecule C is specifically engineered to facilitate the attachment of a peptide to an amino group of a solid resin within an aqueous solution. Its structure comprises:
Fmoc-AA-CH2-Ph-Rx-COOH (FIG. 3)

The Fmoc group, which stands for 9-Fluorenylmethyloxycarbonyl group, is employed as a protecting group for amino groups of amino acids, AA denotes the initial amino acid in the peptide, Ph represents the phenyl group, and —COOH denotes the carboxyl group. The carboxyl group of initial amino acid is linked to the phenyl group through a benzylic ester (—COO—CH2-Ph-). This linkage can be selectively cleaved under strong acidic conditions at the end of the peptide synthesis process. Consequently, the peptide will be liberated from linker molecule C in this acidic environment,

Molecule C can be linked to the amino group on solid resins, such as amino peg resin, to form an amide bond by using a coupling agent like EDC (1-Ethyl-3-(3-dimethylaminopropyl) carbodiimide) in aqueous solution.

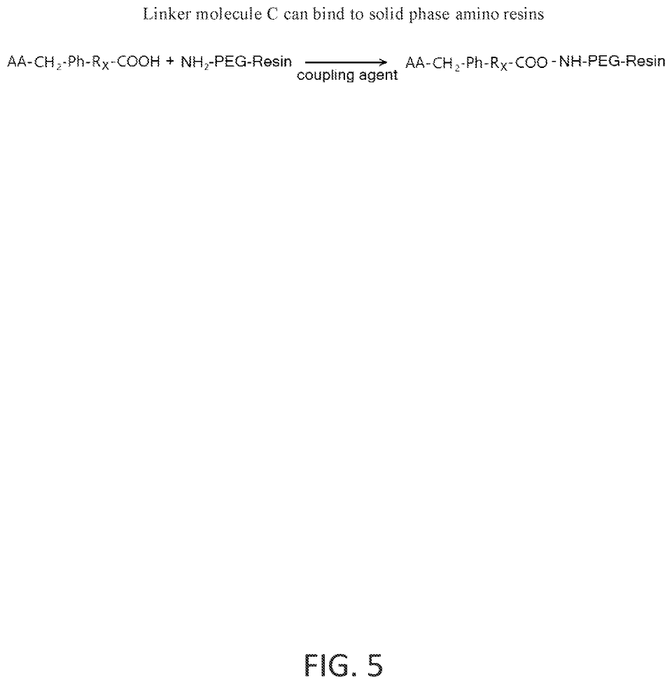

Fmoc-AA-CH2-Ph-Rx-COOH+NH2-solid resin+coupling agent→Fmoc-AA-CH2-Ph-Rx-COO—NH2-solid resin in an aqueous solvent (FIG. 5)

The Fmoc group is attached to the N-terminal end of the amino acid, while Fmoc-AA-CH2-Ph-Rx-COOH is linked to the amino group on hydrophilic resins like amino peg resin. This linker molecule C, acts as an intermediary between the Fmoc Amino Acid and the solid resin by forming amide bond between-COOH of Linker molecule C and NH2-resin by coupling agent DEC in an aqueous solvent. The structure of this linker molecule C closely resembles that of the linker molecule A. The main difference is that linker molecule A is attached to the solid phase via an ionic bond, whereas Linker Molecule C is connected to an amino resin through an amide bond, which is formed between carboxyl group in linker molecule C and amino group of amino resin by using the coupling agent DBC in an aqueous solvent. Similar to linker molecule A, linker molecule C can be cleaved under acidic conditions upon the conclusion of the peptide synthesis, facilitating the release of the synthesized peptide. The Rx group includes but not limited to the methoxyphenyl group (—O—CH2-Ph-), methylene group (—CH2-), carboxylate group (—OOC—), carboxymethylene group (—OOC—CH2-), amino group (—NH—), and ether linkage group (—O—). These chemical groups are utilized individually or in combinations of two or more groups in Rx structure. The Rx group comprises two or more carbon atoms, preferably 5 to 25 carbon atoms. The Rx group is a “spacer” to provide space between Ph and COOH, the spacer makes linker molecule flexible, Rx also facilitates binding Fmoc-AA-CH2-Ph- and —COOH group.

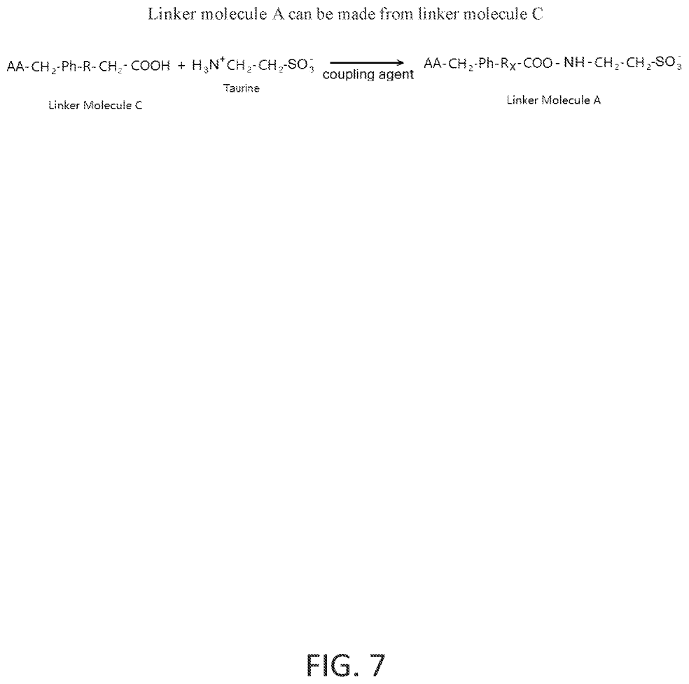

Linker molecule C can also serve as a precursor for linker molecule A. Linker molecule C can couple with 2-aminoethanesulfonic acid (Taurine) to create linker molecule A, First, Fmoc-AA-CH2-Ph-Rx-COOH is combined with Taurine by a coupling agent (EDC) in aqueous solution, subsequently, the Fmoc group can be removed, resulting in the formation of linker molecule A,
Fmoc-AA-CH2-Ph-Rx-COOH+NH2—CH2—CH2-SO₃-(Taurine)→Fmoc-AA-CH2-Ph-Rx-COO—NH—CH2—CH2-SO₃⁻(FIG. 7)

### Linker Molecule D

The linker molecule D specifically designed to facilitate the attachment of a peptide to a carboxyl group on a solid resin within an aqueous solution. Its structure is Fmoc-AA-CH2-Ph-Rx-NH2. The Fmoc group, short for 9-Fluorenylmethyloxycarbonyl, is used as a protective group for the amino groups of amino acids in solid-phase peptide synthesis. AA denotes the initial amino acid in the peptide. Ph represents the phenyl group, and —NH2 denotes the amino group. In this structure, the carboxyl group of an amino acid is connected to the phenyl group via a benzylic ester (—COO—CH2-Ph-). This linkage can be selectively cleaved under strong acidic conditions, at the end of the peptide synthesis process, the peptide is liberated from linker molecule D in acidic environment.

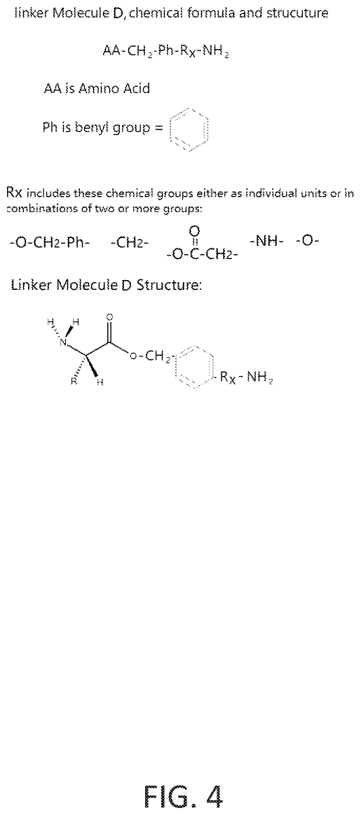

Linker molecule D general structure: Fmoc-AA-CH2-Ph-Rx-NH2 (FIG. 4)

Linker molecule D can be conjugated to the carboxyl group present on solid resins, such as carboxyl methyl cellulose or carboxyl agarose beads, to create an amide bond. This reaction can be facilitated by employing a coupling agent EDC in aqueous solution. —NH2 in linker molecule D and —COOH on the solid resin can be coupled in an aqueous solvent using a coupling agent. This reaction results in the formation of an amide bond connecting the linker molecule D and carboxyl resin.

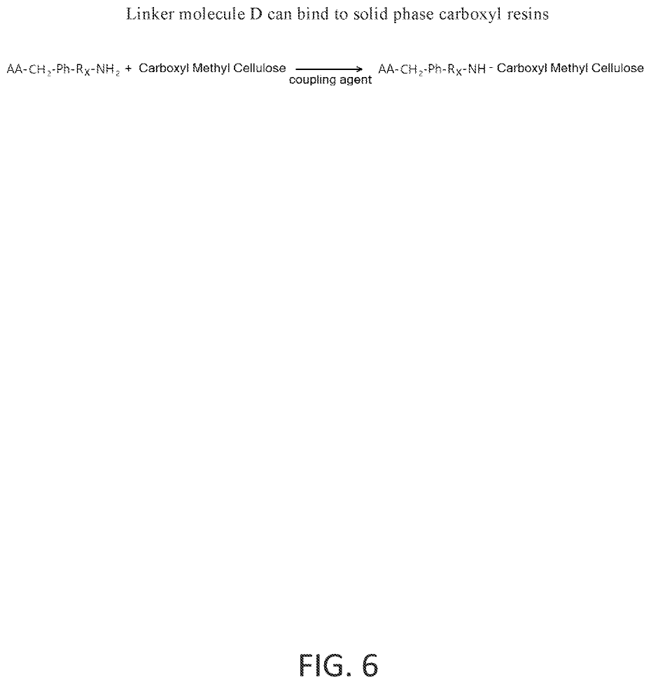

Fmoc-AA-CH2-Ph-Rx-NH2+HOOC-solid resin+coupling agent→Fmoc-AA-CH2-Ph-Rx-NH—OOC-solid resin in an aqueous solvent (FIG. 6)

Linker molecule D is similar to linker molecule B, the key distinction lies in how linker molecule D is attached to the solid phase. In this case, molecule D is linked to the solid phase through the use of a coupling agent like EDC to form an amide bond directly on the solid phase in an aqueous solution. This sets it apart from Linker molecule B. Molecule D also has a cleavable structure —COO—CH2-Ph-, the linker molecule D can be cleaved under acidic conditions to release the peptide chain after it reaches the desired length.

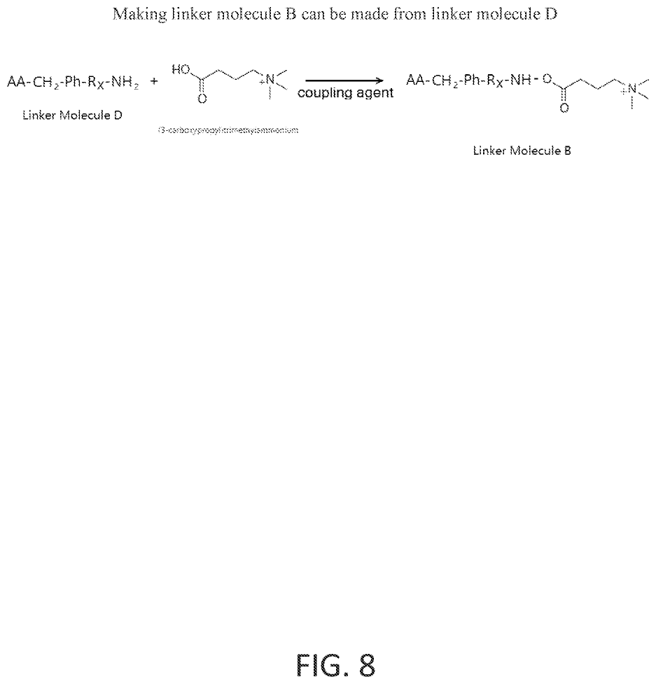

Linker molecule D can also function as a precursor for linker molecule B. Linker molecule D can react with (3-carboxypropyl) trimethylammonium to produce linker molecule B by a coupling agent (EDC) in an aqueous solvent, subsequently, the Fmoc group is removed, resulting in the formation of linker molecule B (FIG. 8).

### A Process for Dissolving Hydrophobic Molecules in Aqueous Solution Specially for Dissolving Fmoc-AA in Aqueous Solution

In the present invention, Fmoc-AA is utilized as a building block for peptide synthesis in an aqueous solution. Fmoc-amino acids (Fmoc-AA) serve as the fundamental building blocks employed in solid-phase peptide synthesis (SPPS) conducted in organic solvents. In Fmoc-based Solid-Phase Peptide Synthesis (SPPS), the peptide chain is constructed sequentially, one amino acid at a time, while attached to an insoluble resin support. Fmoc-amino acids (Fmoc-AA) are extensively employed in the peptide synthesis industry. Nevertheless, it's essential to note that Fmoc-AA is not readily soluble in aqueous solvents. To enable the utilization of Fmoc-AA as a building block in an aqueous solution, a specialized procedure has been developed to render Fmoc-AA soluble in aqueous solvents. The present invention devised a method to render Fmoc-AA soluble in water. First, none ionic surfactant is dissolved in a small amount of water-mixable organic solution, creating solution A, and then secondly, a Fmoc-AA is dissolved in a small amount of water-mixable organic solvent to create solution B, Solution A is thoroughly mixed with Solution B. The resulting mixture is then vigorously added to an aqueous solution. Fmoc-AA aqueous solution is obtained (FIG. 9),

For a clearer understanding of the procedure for dissolving Fmoc-AA in an aqueous solution, a simple example is illustrated here:

4 grams of Tween 80 are added to 2 ml of DMSO to create solution A. and 1 grams of Fmoc-AA are added to 5 ml of DMSO to create solution B, solution A and solution B are mixed thoroughly, and then the resulting mixture is added to 88 ml of water to obtain a 1% concentration of Fmoc-AA in aqueous solution.

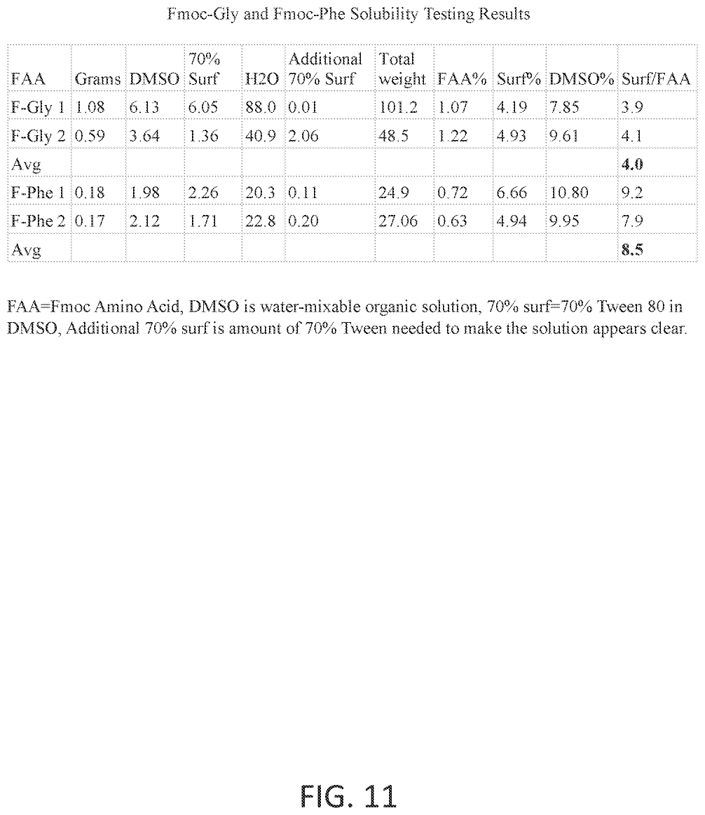

To dissolve water-insoluble Fmoc protected Amino Acid at room temperature, such as Fmoc-Phenylalanine, it typically requires approximately eight times the amount of Tween 80 in an aqueous solution, to dissolve water-soluble Fmoc protected Amino Acid, such as Fmoc-Glycine, it typically requires approximately four times the amount of Tween 80 in an aqueous solution. Fmoc-Gly and Fmoc-Phe solubility test results are listed in FIG. 11.

FIG. 11 presents a table showing test results of the surfactant-to Fmoc-AA ratio (weight to weight) with two different AA, It indicates that, for water soluble amino acid Gly, the optimal ratio of Surfactant to Fmoc-AA is 4:1 (w/w) to achieve soluble in an aqueous solution at room temperature, it indicates that for non-soluble amino acid Phe, the required ratio of surfactant to Fmoc-AA is 8:1 (w/w) to achieve soluble in an aqueous solution.

While augmenting the concentration of surfactants can enhance the Fmoc-AA concentration in an aqueous solution, heightened concentrations may lead to increased viscosity, potentially reducing coupling efficiency. In such instances, elevating the temperature becomes necessary to mitigate solution viscosity and enhance the efficiency of the coupling process. Additionally, an increase in temperature can significantly improve the solubility of Fmoc-AA in the water solution.

In this invention, Fmoc deprotection, amino acid coupling, and peptide cleaving are all carried out in an aqueous solution. The addition of Fmoc-AA is the only step that requires a small amount of water-miscible organic solvent, that significantly reduce the overall consumption of organic solvents.

Deprotection of Fmoc in aqueous solution typically occurs under alkaline conditions with a pH range of 9.5-10.5 by using diluted NaOH or ethyl amine in 10% Tween 80, followed by a washing step in an approximately 10% tween 80, it is necessary to use surfactant aqueous solution since the removed Fmoc is not soluble in pure aqueous solution.

Coupling is facilitated by EDC at a pH range of 4-5. First, additional FAA dissolved in an aqueous solution is introduced, followed by the addition of a coupling agent such as EDC. Coupling is performed within a pH range of 4-5 for a duration of 20-30 minutes. It may be necessary to increase the temperature for faster coupling, especially when dealing with non-water-soluble amino acids. Additionally, the percentage of surfactant needed may vary depending on the solubility of the amino acids. It's important to note that higher surfactant levels may increase viscosity, so temperature adjustments may be required to reduce viscosity and increase coupling efficiency.

Process to Synthesis Peptide in Aqueous Solution with Four Different Hydrophilic Resins

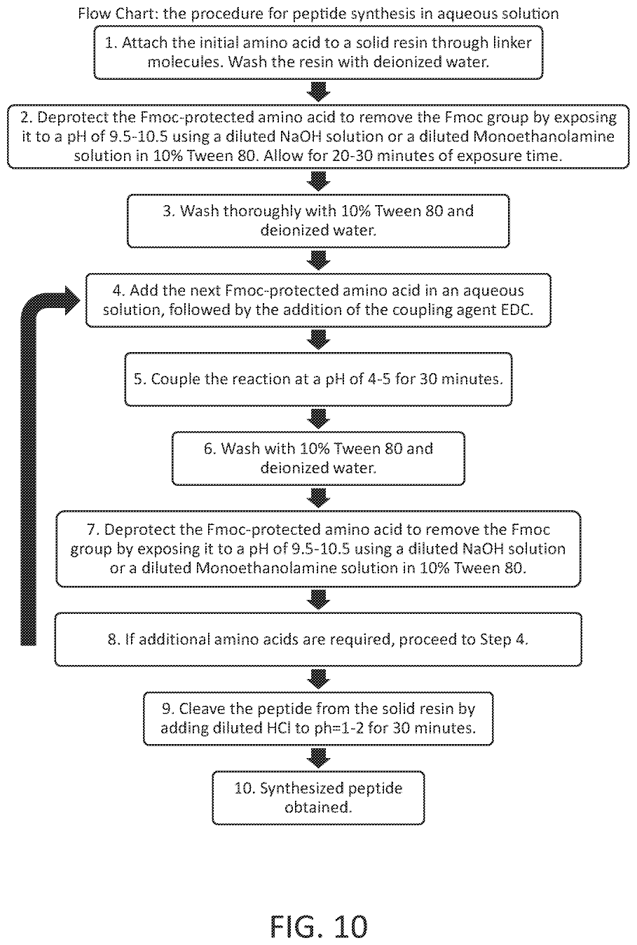

Anionic exchange resin and cationic exchange resin are commonly utilized in water purification, while carboxyl cellulose and amino resin find wide applications in biotechnology. This embodiment leverages these four hydrophilic resins to facilitate solid-phase peptide synthesis (SPPS) in an aqueous solution. The synthesis of peptides in this process follows a series of steps, as depicted in the flowchart FIG. 10. The initial amino acids are initially immobilized onto solid supports by linker molecules. This immobilization is achieved by attaching the initial amino acids to the solid resin through linker molecules.

Linker molecule A is employed to immobilize AA onto an anionic exchange resin as a solid phase, linker molecule B is used to attach AA to cationic resin, linker molecule C facilitates the connection of AA to amino resin, and linker molecule D is responsible for binding AA to carboxyl resin. AA are initial Amino Acids in peptide synthesis. As all four linker molecules are hydrophobic due to the benzylic ester structures in these four linker molecules, it is essential to dissolve them in an aqueous solution following the process for dissolving hydrophobic molecules in aqueous solution before employing these four linker molecules to bind hydrophilic solid phase resins in aqueous solution.

### Procedure for Employing an Anion Exchange Resin as the Solid Phase in Solid-Phase Peptide Synthesis (SPPS)

The initial step entails dissolving linker molecule A in aqueous solution, following this, the linker molecule A solution is incubated with an anionic exchange resin for several hours at room temperature for attaching linker molecule A to anionic exchange resin. Afterward, it is washed 10% Tween 80 solution and with DI water, preparing it for coupling with next additional Fmoc-AA. In most cases, Amino acid (AA) in Linker Molecule A is also Fmoc protected with structure:
Fmoc-AA-CH2-Ph-Rx-SO3, AA is initial amino acid

If the initial amino acid in linker molecule A is Fmoc-protected, following the attachment of linker molecule A to anionic resin, the Fmoc protective group must be removed by exposing it to a solution with pH range of 9.5-10.5 for 30-60 minutes in a 10% Tween 80 solution, followed by rinsing with DI water. At this point, it is ready for coupling with additional Fmoc-AA.

### Procedure for Employing Cationic Exchange Resin as Solid Phase

When using a cationic exchange resin for SPPS aqueous solution, linker molecule B is employed to attach the initial amino acid to the solid resin, similar to the procedure for using an anionic exchange resin. First, linker molecule B is dissolved in an aqueous solution. Subsequently, it is incubated with the cationic exchange resin for several hours and then washed with DI water. In cases where the amino acid is protected by Fmoc, deprotection is achieved by exposing it to a pH of 9.5-10.5 solution, followed by a wash with a 10% Tween 80 solution and DI water. After these steps, the system is ready for adding additional AA. Fmoc protected Linker molecule B structure is this: Fmoc-AA-CH2-Ph-Rx-N(CH3)₃+

### Procedure for Using Amino Resins as Solid Phase

Linker Molecule C: Fmoc-AA-CH2-Ph-Rx-COOH is designed to bind AA to amino resins such as amino PEG resin which is often used in biologic lab. Linker molecule C contains a carboxyl group capable of binding to the amino group of an amino resin, such as amino PEG resin, through coupling agent like EDC. Linker molecule C is dissolved in an aqueous solution. The resulting Linker molecule C solution is then incubated with the amino resin, followed by the addition of the coupling agent EDC. This facilitates the formation of an amide bond between the carboxyl group in Linker molecule C and the amino group on the resin.

The coupling reaction occurs at a pH of 4-5 and room temperature, lasting for 30-60 minutes. Subsequently, any unreacted amino groups on the resin are capped by forming an amide bond with acidic acid. And deprotection is achieved by removing the Fmoc group at a pH of 9.5-10.5. The system is then washed with a 10% T solution and DI water, preparing it for the addition of the next AA to the initial amino acid.

### Procedure for Using Carboxyl Resins as Solid Phase

Linker Molecule D: Fmoc-AA-CH2-Ph-Rx-NH2 features an —NH2 group capable of binding to the carboxyl group of a carboxyl resin through coupling with the assistance of a coupling agent like EDC. For example, carboxyl resin such as carboxyl methyl cellulose resin can be used. To render linker molecule D soluble in an aqueous solution, the process for dissolving hydrophobic molecules is employed. The coupling of —NH2 and carboxyl group occurs at a pH of 4-5 and room temperature, lasting for 30-60 minutes. Afterward, any unreacted carboxyl groups on the resin are capped by forming an amide bond with a primary amine. Deprotection is achieved by removing the Fmoc group at a pH of 9.5-10.5. The system is then washed with a 10% Tween 80 solution and DI water, preparing it for the addition of the next amino acid (AA) to the initial amino acid.

### Procedure for Peptide Synthesis in Aqueous Solution

Attach the initial amino acid to the solid phase. Following the procedure described earlier for linking the initial amino acid to solid resins through linker molecules, proceed to the next step.

Deprotection involves washing the solid phase with deionized water, followed by the removal of Fmoc. Achieve Fmoc deprotection by maintaining a pH range of 9.5-10.5, using a diluted NaOH solution or a diluted monoethanolamine solution in 10% Tween 80. Subsequently, perform sequential washing with 10% Tween 80 and deionized water.

Wash off excess agents by rinsing with 10% Tween 80 and deionized water to remove Fmoc from the solution.

Introduce the next Fmoc-AA. Begin by dissolving Fmoc-AA in an aqueous solution, using the process for dissolving hydrophobic molecules. Subsequently, merge this Fmoc-AA solution with the solid resin previously attached to the linker molecules, either with the initial amino acid or as part of the growing peptide chain.

Couple Fmoc-AA to the preceding amino acid in the peptide chain using a coupling agent such as EDC at pH 4-5 and room temperature in a 10% Tween 80 solution for 30 minutes. Elevating the temperature can enhance the efficiency of the coupling reaction.

Following the coupling reaction, eliminate excess Fmoc-AA and EDC by sequentially washing with 10% Tween 80 and deionized water. It is essential to include a wash with 10% Tween 80, as Fmoc is not soluble in the aqueous phase.

Deprotection involves washing the solid phase with deionized water, followed by the removal of Fmoc. Achieve Fmoc deprotection by maintaining a pH range of 9.5-10.5, using a diluted NaOH solution or a diluted monoethanolamine solution in 10% Tween 80. Subsequently, perform sequential washing with 10% Tween 80 and deionized water.

If additional amino acids are needed, return to step 4 and repeat steps 4 through 7 until the desired peptide chain is established. Proceed to the next step thereafter.

Cleave the linker molecule under acidic conditions. Utilize diluted HCl or diluted H3PO4 to maintain a pH of 1-2, releasing the peptide from the solid phase through acidic cleavage.

Preparing Linker Molecule a from Linker Molecule C

Linker molecule A can be synthesized by bonding Linker Molecule C with 2-aminoethanesulfonic acid (Taurine). Initially, dissolve Linker molecule C in an aqueous solution. Add taurine and EDC (1-ethyl-3-(3-dimethylaminopropyl) carbodiimide) to this solution at a pH range of 4 to 5. Maintain the reaction mixture at room temperature for 60 minutes. Following this coupling reaction, obtain Linker molecule A (as illustrated in FIG. 7).

Preparing Linker Molecule B from Linker Molecule D

Linker molecule B can be created by combining Linker Molecule D with CTMA (carboxypropyl) trimethylammonium). Start by dissolving Linker molecule D in an aqueous solution. Introduce CTMA and EDC (1-ethyl-3-(3-dimethylaminopropyl) carbodiimide) into this solution within a pH range of 4 to 5. Allow the reaction mixture to be maintained at room temperature for 60 minutes. After the completion of this coupling reaction, obtain Linker molecule B (as illustrated in FIG. 8).

The procedure for improving the solubility of hydrophobic molecules. The procedure for improving the solubility of hydrophobic molecules in an aqueous solution is illustrated by dissolving two Fmoc-protected amino acids: Fmoc-protected Glycine and Fmoc-protected Phenylalanine. While amino acid Gly is water-soluble, amino acid Phe exhibits poor solubility in an aqueous solution. Fmoc itself is very poor solubility in aqueous solution. It's worth noting that both Fmoc-Gly and Fmoc-Phe display poor solubility in aqueous solutions.

The following steps outline the process by which two Fmoc-protected amino acids, Fmoc-Glycine (Fmoc-Gly), and Fmoc-Phenylalanine (Fmoc-Phe), attain solubility in an aqueous solution.

### Dissolving Fmoc-Gly in Aqueous Solution

Step 1: Dissolve 1.08 grams of Fmoc-Gly in 6.13 ml of DMSO solvent. Next, add 6.00 milliliters (mL) of a 70% Tween 80 in DMSO solution to the Fmoc-Gly DMSO solution.
Step 2: Pour this mixture into 87 mL of deionized (DI) water. Initially, the solution may appear slightly cloudy.
Step 3: Gradually, add 70% Tween 80 dropwise until the solution becomes clear. After adding 0.01 mL of 70% T, the solution becomes clear.
Step 4: This process yields a solution containing 1.07% Fmoc-Gly in an aqueous solution with 7.85% DMSO. The ratio of Tween 80 to Fmoc-Gly is approximately 4:1 (w/w), as illustrated in FIG. 11.

### Dissolving Fmoc-Phe in Aqueous Solution

Dissolve 0.18 grams of Fmoc-Phe in 1.98 DMSO of solvent. Next, add 2.26 milliliters (mL) of a 70% Tween 80 in DMSO solution to Fmoc-Phe solution.

Transfer this mixture into 20.38 mL of deionized water. Initially, the solution appears cloudy.

Gradually, add 70% T dropwise until the solution becomes clear. An additional 0.11 mL of 70% Tween 80 is required for the solution becomes clear.

This process yields a solution containing 0.72% Fmoc-Phe in an aqueous solution with 10.80% DMSO. The ratio of Tween 80 to Fmoc-Phe is approximately 8:1 (w/w), as shown in FIG. 11.

Raising the temperature can lead to significantly increase the solubility of Fmoc-Phe in aqueous solution.

Solid Phase Synthesis Dipeptide his-Gly in Aqueous Solution with Anionic Exchange Resin as Solid Phase

Linker molecule A has structure of AA-CH2-Ph-Rx-SO3⁻, here AA shall be Gly, that is Gly-CH2-Ph-Rx-SO₃⁻. Attaching linker molecule A to an anionic exchange resin is achieved by dissolving 0.2 grams of linker molecule A with Gly as the initial amino acid in a 20 mL aqueous solution, employing the process for dissolving hydrophobic molecules in an aqueous solvent. The resulting mixture is then incubated with one gram of an anionic exchange resin for one hour, ensuring constant agitation through vibration or stirring. (In this context, the anionic exchange resin features quaternary ammonium cations).

To deprotect the Fmoc group, maintain a pH range of 9.5-10.5 using a diluted NaOH solution or a diluted Monoethanolamine solution in 10% Tween 80, followed by sequential washing with 10% Tween 80 and DI water.

Wash the mixture with a 10% Tween 80 solution and then with deionized (DI) water, respectively.

Subsequently, introduce 0.3 grams of an Fmoc-Histidine aqueous solution to the anionic exchange resin attached by linker molecule A with Gly as the initial amino acid, ensuring continuous agitation through vibration and stirring

Coupling Fmoc-Histidine with Gly in the linker A attached to anionic exchange resin by using a coupling agent such as EDC at pH 4-5 and room temperature in a 10% Tween 80 solution for 30 minutes.

Perform a thorough washing with a 10% Tween 80 solution followed by DI water.

To deprotect the Fmoc group, maintain a pH range of 9.5-10.5 using a diluted NaOH solution or a diluted Monoethanolamine solution in 10% Tween 80, followed by sequential washing with 10% Tween 80 and DI water.

Now we have a dipeptide His-Gly on the anionic exchange resin.

To release this di-peptide into an aqueous solution, cleave this dipeptide with diluted HCl at pH=1.

The anionic exchange resin can be regenerated by standard regeneration process for ionic exchange procedure.

### Using Carboxyl Methyl Cellulose as Solid Phase for Synthesis Peptide

One gram of linker molecule C was dissolved in 20 of aqueous solution. Subsequently, 5 grams of carboxyl methyl cellulose were added, along with one gram of EDC. The mixture was allowed to react for one hour at a pH of 4-5. Following this, a washing step was performed using a 10% Tween 80 solution and deionized (DI) water.

Before deprotection Fmoc, it is preferred to cap any unreacted carboxyl groups on the solid phase. This can be achieved by introducing 0.1 gram of monoethanolamine and additional 0.5 EDC at pH=4-5 to facilitate the reaction with any non-reacted carboxyl groups on the carboxyl methyl cellulose (warning: the monoethanolamine needs to be neutralized before added to avoid pH value jump above 9). The resulting mixture should be washed again with a 10% Tween 80 solution and DI water.

After the washing steps, the deprotection process can be carried out by adding diluted NaOH or piperidine at pH around 9.5-10.5 to keep it for 30 minutes. Once this step is completed, the material is ready for further peptide synthesis processing.

Making Linker Molecule a from Linker Molecule C, an Example

Dissolve Linker Molecule C in an aqueous solution. Add an excess amount of Taurine with a molarity ratio of Taurine to C at 2:1. Then, introduce EDC at a ratio of 1:1 (Taurine to EDC) at room temperature with a pH range of 4-5. Allow the reaction to proceed for 60 minutes (FIG. 7)

Linker molecule A is soluble in organic solvent meanwhile, Taurine is very poor soluble in DMSO, ethanol, ether, acetone, and methanol. To remove the excess Taurine, add an organic solvent such as ethanol to precipitate Taurine out of the solution. Taurine can interfere with the peptide synthesis process. After precipitation out of Taurine, remove extra the organic solvent under vacuum.

The resulting solution, containing Linker Molecule A with Fmoc-AA in one end of Linker molecule A, is now ready for binding to an anionic exchange resin for the synthesis process.

Although the invention has been explained in relation to its preferred embodiment, it is to be understood that many other possible modifications and variations can be made without departing from the spirit and scope of the invention.

1. A linker molecule consisting of:
a chemical structure of AA-CH2-Ph-Rx-SO₃⁻,
wherein a sulfonate group is found at one end of said chemical structure;
wherein said sulfonate group can form an ionic bond with an anionic exchange resin in an aqueous solution;
wherein an AA group is an amino acid selected from the group consisting of alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine;
wherein said amino acid is found at one end of said chemical structure opposite said sulfonate group;
wherein said amino acid is connected to said chemical structure with a benzylic ester bond of —O—CH2-Ph-;
wherein said benzylic ester bond between said amino acid and said-CH2-Ph-Rx-SO3⁻ is through the C-terminus of said amino acid,
wherein said benzylic ester bond is a cleavable bond that is susceptible to acidic conditions;
wherein an Rx group facilitates a connection between the benzene group and said sulfonate group;
wherein an Rx group comprises structure of:
Rx=—CH₂-Phx-(HN—CH₂—CH₂—NH) y-(COO—CH₂—CH₂—COO) z-NH—(—CH₂) n-
wherein an Phx is A variable phenyl structure where X=0 to 1, indicating the possible presence or absence of a phenyl group,
wherein an diamine structure comprises structure of (HN—CH₂—CH₂—NH) y, where Y=0 to 1, representing the optional inclusion of a diamine moiety.
wherein an diacidic structure comprises structure of (COO—CH₂—CH₂—COO) z-, where Z=0 to 1, allowing for the inclusion or exclusion of a diacidic moiety. This diacidic molecule binds amines by amide bonds.
wherein a linear methylene chain structure comprises (—CH₂—) n, where n=2 to 10, providing variable chain length.
wherein the linker molecule is developed for tethering amino acids to an anionic exchange resin in an aqueous solution, facilitating solid-phase peptide synthesis in aqueous solution.

2. The linker molecule of claim 1,
wherein said Rx group further comprises a methoxyphenyl group of —O—CH2-Ph-.

3. The linker molecule of claim 1,
wherein said Rx group is a chain of methylene groups of [—CH2-]—(CH2) n-; and
wherein n is a number between two and ten.

4. The linker molecule of claim 1,
wherein said Rx group further comprises one to three carboxylate groups of —OOC—.

5. The linker molecule of claim 1,
wherein said Rx group further comprises one to three carboxymethylene group of —OOC—CH2-.

6. The linker molecule of claim 1,
wherein said Rx group further comprises one to three amino group of —NH—.

7. The linker molecule of claim 1,
wherein said Rx group further comprises one to ten ester linkage group of —O—.

8. A linker molecule comprising:
a chemical structure of AA-CH2-Ph-Rx-N(CH3)₃⁺,
wherein a trimethylammonium group is found at one end of said chemical structure;
wherein said trimethylammonium group can form an ionic bond with a cationic exchange resin in an aqueous solution;
wherein an amino acid is found at one end of said chemical structure opposite said trimethylammonium group;
wherein said amino acid is connected to said chemical structure with a benzylic ester bond of —O—CH2-Ph-;
wherein said benzylic ester bond is a cleavable bond that is susceptible to acidic conditions;
wherein an Rx group facilitates a connection between the benzene group and said trimethylammonium group;
wherein said Rx group comprises two or more carbon atoms;
wherein the linker molecule is developed for tethering amino acids to a cationic exchange resin in an aqueous solution, facilitating solid-phase peptide synthesis in aqueous solution.

9. The linker molecule of claim 8,
wherein said Rx group further comprises a methoxyphenyl group of —O—CH2-Ph-.

10. The linker molecule of claim 8,
wherein said Rx group further comprises one to ten methylene groups of —CH2-.

11. The linker molecule of claim 8,
wherein said Rx group further comprises one to three carboxylate groups of —OOC—.

12. The linker molecule of claim 8,
wherein said Rx group further comprises one to three carboxymethylene group of —OOC—CH2-.

13. The linker molecule of claim 8,
wherein said Rx group further comprises one to three amino group of —NH—.

14. The linker molecule of claim 8,
wherein said Rx group further comprises one to ten ester linkage group of —O—.

15. A linker molecule comprising:
a chemical structure of Fmoc-AA-CH2-Ph-Rx-COOH,
wherein a carboxyl group is found at one end of said chemical structure;
wherein said carboxyl group can form an amide bond with an amino group of an amino resin with the help of a coupling agent in an aqueous solution;
wherein a Fmoc-amino acid is found at one end of said chemical structure opposite said carboxyl group;
wherein said Fmoc-amino acid is connected to said chemical structure with a benzylic ester bond of —O—CH2-Ph-;
wherein said benzylic ester bond is a cleavable bond that is susceptible to acidic conditions;
wherein an Rx group facilitates a connection between the benzene group and said carboxyl group;
wherein said Rx group comprises two or more carbon atoms;
wherein the linker molecule is developed for attaching amino acids to an amino resin in an aqueous solution, facilitating solid-phase peptide synthesis in aqueous solution.

16. The linker molecule of claim 15,
wherein said Rx group further comprises a methoxyphenyl group of —O—CH2-Ph-.

17. The linker molecule of claim 15,
wherein said Rx group further comprises one to ten methylene groups of —CH2-.

18. The linker molecule of claim 15,
wherein said Rx group further comprises one to three carboxylate groups of —OOC—.

19. The linker molecule of claim 15,
wherein said Rx group further comprises one to three carboxymethylene group of —OOC—CH2-.

20. The linker molecule of claim 15,
wherein said Rx group further comprises one to three amino group of —NH—.

21. The linker molecule of claim 15,
wherein said Rx group further comprises one to ten ester linkage group of —O—.

22. A linker molecule comprising:
a chemical structure of Fmoc-AA-CH2-Ph-Rx-NH2,
wherein an amino group is found at one end of said chemical structure;
wherein said amino group can form an amide bond with a carboxyl group of a carboxyl resin with the help of a coupling agent in an aqueous solution;
wherein a Fmoc-amino acid is found at one end of said chemical structure opposite said amino group;
wherein said Fmoc-amino acid is connected to said chemical structure with a benzylic ester bond of —O—CH2-Ph-;
wherein said benzylic ester bond is a cleavable bond that is susceptible to acidic conditions;
wherein an Rx group facilitates a connection between the benzene group and said amino group;
wherein said Rx group comprises at least two carbon atoms;
wherein the linker molecule is developed for attaching amino acids to a carboxyl resin in an aqueous solution, facilitating solid-phase peptide synthesis in aqueous solution.

23. The linker molecule of claim 22,
wherein said Rx group further comprises a methoxyphenyl group of —O—CH2-Ph-.

24. The linker molecule of claim 22,
wherein said Rx group further comprises one to ten methylene groups of —CH2-.

25. The linker molecule of claim 22,
wherein said Rx group further comprises one to three carboxylate groups of —OOC—.

26. The linker molecule of claim 22,
wherein said Rx group further comprises one to three carboxymethylene group of —OOC—CH2-.

27. The linker molecule of claim 22,
wherein said Rx group further comprises one to three amino group of —NH—.

28. The linker molecule of claim 22,
wherein said Rx group further comprises one to ten ester linkage group of —O—.

29. A method of dissolving hydrophobic molecules into aqueous solution to facilitate solid-phase peptide synthesis in aqueous solution comprising:
a first step of a non-ionic surfactant dissolved in water-mixable organic solution, creating a first solution;
a second step of a hydrophobic molecule comprising a chemical structure of Fmoc-AA is dissolved in water mixable organic solvent, creating a second solution;
a third step of mixing said first solution and said second solution, creating a mixture;
a fourth step of adding said mixture into aqueous solution, creating a resulting 1-20% Fmoc-AA aqueous solution.
